(19) United States
(12) Patent Application Publication
(10) Pub. No.: US 2005/0008633 A1
(43) Pub. Date: Jan. 13, 2005

(54) Chemical and physical modulators of bioavailability of inhaled compositions

(75) Inventors: Rita Vanbever, Brussels (BE); David A. Edwards, Boston, MA (US)

Correspondence Address: ELMORE CRAIG, P.C., 209 MAIN STREET, N. CHELMSFORD, MA, 01863, US

(73) Assignees: Advanced Inhalation Research Inc., Cambridge, MA (US); Universite Catholique de Louvain, Louvain-La-Neuve (BE)

(21) Appl. No.: 10/843,693

(22) Filed: May 11, 2004

Related U.S. Application Data
(60) Provisional application No. 60/471,645, filed on May 19, 2003.

(51) Int. Cl. A61K 39/395

(52) U.S. Cl. 424/130.1; 514/2

(57) ABSTRACT

The present invention is drawn to methods of enhancing bioavailability, pulmonary absorption and/or optimal dosing of a biologically active agent. The methods of the invention enhance the pulmonary absorption and bioavailability of a biologically active agent by administering to the pulmonary system of a subject, a biologically active agent and a macrophage inhibiting agent that is suitable for administration to a subject's pulmonary system.

### RELATED APPLICATIONS

This application claims the benefit of U.S. Provisional Application No. 60/471,645, filed on May 19, 2003. The entire teachings of the above application is incorporated herein by reference.

### BACKGROUND OF THE INVENTION

Unique physiological features make the lungs an attractive port of entry to the systemic circulation for the administration of drugs. For example, i) the alveoli present a large surface area for absorption (≈100 m²) that is immediately accessible to drugs or biologically active agents; ii) there is only a thin diffusion path that separates the airspace from the bloodstream (the alveolar epithelium, the vascular epithelium and their respective basal membranes are less than 0.5 μm thick in parts; iii) the high blood flow rate (≈5 L/min) of the pulmonary circulation rapidly distributes molecules throughout the body, without first-pass hepatic metabolism; and iv) metabolic activity in the lung is relatively low (Komada, F., et al., “Intratracheal delivery of peptide and protein agents: Absorption from solution and dry powder by rat lung,” J. Pharm. Sc. 83:863-867 (1994)).

Inhalation aerosols can offer significant potential for non-invasive systemic administration of peptide and protein therapeutics (Adjei, A. L., and Gupta P. K., Inhalation delivery of therapeutic peptides and proteins. New York-Basel-Hong Kong: Marcel Dekker Inc. (1997); Owens, D. R., “New horizons—Alternative routes for insulin therapy,” Nat. Rev. Drug Discov., 1:529-540 (2002)). Yet, low bioavailabilities result from uncontrolled biological losses of molecules in the airway lumen and/or respiratory tissue and considerably diminish efficiency (Owens, D. R., Nat. Rev. Drug Discov., 1:529-540 (2002)). For example, insulin (5.8 kDa) inhaled into the human lungs using high technology inhaler devices or engineered particles is absorbed and appears in the bloodstream within minutes with an average bioavailability of 10% relative to subcutaneous injection (Owens, D. R., supra; Skyler J. S., et al., “Efficacy of inhaled human insulin in type 1 diabetes mellitus: a randomised proof-of-concept study,” Lancet, 357:331-335 (2001); Brunner, G. A., et al., “Dose-response relation of liquid aerosol inhaled insulin in type I diabetic patients,” Diabetologia, 44:305-308 (2001)). Large proteins, like immunoglobulin G (150 kDa), absorb into the bloodstream following intratracheal instillation in animals over many hours, often with bioavailabilities that can be significantly lower than 5% (Folkesson, H. G., et al., “Permeability of the respiratory tract to different sized macromolecules after intratracheal instillation in young and adult rats,” Acta Physiol. Scand., 139:347-354 (1990); Komada, F., et al., J. Pharm. Sc., 83:863-867 (1994)).

The exact nature of protein absorption through the alveolar epithelium into the bloodstream is unclear, although it appears that most protein absorption occurs in the large and highly vascularized alveolar region and that the alveolar epithelium has an important role in the passage of proteins into the bloodstream (Wall, D. A., “Pulmonary absorption of peptides and proteins,” Drug Deliv., 2:1-20 (1995); Folkesson, H. G., et al., “Alveolar epithelial clearance of protein,” J. Appl. Physiol., 80:1431-1445 (1996); Patton, J. S., “Mechanisms of macromolecule absorption by the lungs,” Adv. Drug Delivery Rev., 19:3-36 (1996); Wolff, R. K., “Safety of inhaled proteins for therapeutic use.,” J. Aerosol Med., 11:197-219 (1998)).

Thus, in view of the low bioavailability of many pulmonarily-delivered agents, a need exists for a method of enhancing the bioavailability and pulmonary absorption of agents that are delivered to the pulmonary system. A need also exists for optimizing dosing of agents that are administered to the pulmonary system of a subject.

### SUMMARY OF THE INVENTION

In various embodiments, the present invention is drawn to methods of administering a biologically active agent to the pulmonary system of a subject, enhancing bioavailability and pulmonary absorption of a pulmonarily-delivered biologically active agent, and/or optimizing dosing of a biologically active agent.

In one aspect, the invention is a method of administering a biologically active agent to the pulmonary system of a subject comprising administering a therapeutically active agent and a macrophage inhibiting agent suitable for administration to a pulmonary system.

In another aspect, the invention is a method of enhancing the bioavailability of a biologically active agent by administering to the pulmonary system of a subject, a biologically active agent and a macrophage inhibiting agent that is suitable for administration to a subject's pulmonary system. The macrophage inhibiting agent is present in an amount sufficient to enhance bioavailability of the biologically active agent.

In another aspect, the invention is a method of enhancing the pulmonary absorption of a biologically active agent by administering to the pulmonary system of a subject, a biologically active agent and a macrophage inhibiting agent that is suitable for administration to a subject's pulmonary system.

In another aspect, the invention is a method of enhancing the bioavailability of a biologically active agent that is administered to the pulmonary system of a subject. In this method, the rate of transport of the biologically active agent through the cell linings of the pulmonary system (K(1)) and the rate of degradation of the biologically active agent in the pulmonary system (K(2)) are determined and a dose of the biologically active agent is administered wherein K(1) is at least 5 times greater than K(2). In another embodiment, this method further comprises determining the rate of release of the biologically active agent from a delivery vehicle (K(0)) and administering a dose of said biologically active agent wherein K(0) is less than or equal to K(1).

In another aspect, the invention is a method of determining an optimal dosing regimen for pulmonary delivery of a biologically active agent. K(1) and (K2) are determined and a dosing regimen of the biologically active agent is selected wherein K(1) is at least 5 times greater than K(2).

In still another aspect, the invention is a method of conducting and optimizing dosing for pulmonary delivery of a biologically active agent. K(1) and (K2) are determined and a dosing regimen of the biologically active agent is selected wherein K(1) is at least 5 times greater than K(2).

### BRIEF DESCRIPTION OF THE DRAWINGS

FIG. 1 is a graph depicting the pharmacokinetic properties of pulmonary immunoglobulin G (IgG) in rats with variable number of alveolar macrophages (AM).

FIG. 2 is a graph depicting the pharmacokinetic properties of pulmonary human chorionic gonadotrophin (hCG) in AM-depleted and control rats.

FIG. 3 is a graph depicting the pharmacokinetic properties of pulmonary insulin in AM-depleted and control rats.

FIGS. 4A and 4B are graphs depicting the pharmacokinetic properties of pulmonary and intravenous administration of IgG at variable doses.

### DETAILED DESCRIPTION OF THE INVENTION

The features and other details of the invention, either as steps of the invention or as combination of parts of the invention, will now be more particularly described with reference to the accompanying drawings and pointed out in the claims. It will be understood that the particular embodiments of the invention are shown by way of illustration and not as limitations of the invention. The principle feature of this invention may be employed in various embodiments without departing from the scope of the invention.

The present invention is drawn to methods of administering a biologically active agent to the pulmonary system of a subject, enhancing bioavailability and pulmonary absorption, and/or optimizing dosing of a biologically active agent.

In one aspect, the invention is a method of enhancing the bioavailability of a biologically active agent by administering to the pulmonary system of a subject, a biologically active agent and a macrophage inhibiting agent that is suitable for administration to a subject's pulmonary system. The macrophage inhibiting agent is present in an amount sufficient to enhance bioavailability of the biologically active agent.

As used herein, a “macrophage inhibiting agent” refers to a compound that is able to decrease the uptake of an agent (e.g., biologically active agent) by macrophages (e.g., alveolar macrophages). For example, macrophage inhibiting agents include bisphosphonate compounds (e.g., dichloromethylene diphosphonate, alendronate, risendronate, pamidronate, etidronate, tiludronate). Bisphosphonates are a group of synthetic pyrophosphates characterized by a P—C—P type backbone and can be generally represented by Formula I: 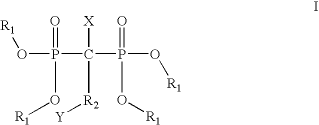
wherein,
R₁ is independently, H, alkyl, aryl or heteroaryl;
X is H, —OR₁ or halogen;
R₂ is H, O, S, N, (CH₂)ₙ, branched alkylene, branched or straight alkenylene or alkynylene;
n is an integer from about 0 to about 18;
Y is H, R₁, halogen, amino, cyano or amido group.

As used herein, “alkyl” refers to a straight chain or branched, substituted or unsubstituted C₁-C₁₈ hydrocarbon group. Examples of suitable alkyl groups include, but are not limited to, methyl, ethyl, propyl, butyl, pentyl, isopropyl, isobutyl, and tert-butyl. As used herein, “halogen” refers to chlorine, bromine, iodine and fluorine. The term “aryl” as used herein refers to unsubstituted and substituted aromatic hydrocarbons. The term “heteroaryl” as used herein refers to unsubstituted or substituted aryl groups wherein at least one carbon of the aryl group is replaced with a heteroatom (e.g., N, O or S). Suitable substituents, include, for example, but are not limited to, halogen, —OH, alkoxy, amino, amido, —SH, cyano, —NO₂, —COOH, —COH, —COOR₁.

Bisphosphonates suitable for use in the invention include those described in U.S. Pat. No. 4,705,651, U.S. Pat. No. 4,327,039, U.S. Pat. No. 5,312,954 and U.S. Pat. No. 5,196,409 to Breuer et al., U.S. Pat. No. 5,412,141 to Nugent, U.S. Pat. Nos. 4,922,007 and 5,019,651 to Kieczykowski et al., U.S. Pat. No. 5,583,122 to Benedict et al., U.S. Pat. No. 6,080,779 to Gasper et al., U.S. Pat. No. 6,117,856 to Benderman et al., U.S. Pat. No. 6,162,929 to Foricher et al. and U.S. Pat. No. 5,885,473 to Papapoulos et al. the entire content of all of which are hereby incorporated by reference.

In particular embodiments, the methods of the invention utilize a macrophage inhibiting agent which is a bisphosphonate compound that is administered as liposomes. For example, it has been demonstrated that the bisphosphonate compound, dichloromethylene diphosphonate (Cl₂MDP), induces most selectively and most effectively the apoptosis of alveolar macrophages when formulated as liposomes (see, e.g., Berg, J. T., et al., “Depletion of alveolar macrophages by liposome-encapsulated dichloromethylene diphosphonate,” J. Appl. Physiol., 74(6):2812-2819 (1993); Thepen, T., et al., “Alveolar macrophage elimination in vivo is associated with an increase in pulmonary immune response in mice,” J. Exp. Med., 170(2):499-509 (1989)). Thus, while liposomes containing Cl₂MDP undergo phagocytosis by alveolar macrophages (AM), thereby allowing Cl₂MDP to induce apoptosis of the alveolar macrophages, free Cl₂MDP does not easily cross the cell membranes and is quickly cleared from the alveoli. Other appropriate delivery vehicles are described herein and/or are known in the art.

Other macrophage inhibiting agents that are suitable for the methods of the invention are also known in the art. For example, physiological modulators (e.g., amino acids) are able to decrease uptake of an agent (e.g., biologically active agent) by macrophages (e.g., alveolar macrophages) (see, e.g., Besterman, J. M., et al., J. Cell Biol., 96(6):1586-1591 (1983); Kooistra, T., et al., Biosc. Rep., 1:587-594 (1981)).

In addition, macrophage uptake (e.g., alveolar macrophage uptake) may also be mediated by non-specific adsorptive endocytosis (see, e.g., Kooistra, T., et al., Biosci. Rep., 1(7):587-594 (1981)). Therefore, compounds that compete with biologically active agents for binding to the plasma membrane of macrophages (e.g., alveolar macrophages) can be used as macrophage inhibiting agents in the methods of the invention. For example, in vitro studies have demonstrated that serum components decrease protein uptake by competing for binding to macrophages (Kooistra, T., et al., supra). Administration of particular compositions, e.g., large porous particles, could also protect the biologically active agent from local phagocytic degradation by releasing the agent at a rate that is slightly less than the rate of pulmonary absorption (see, e.g., Edwards, D. A., et al., “Large porous particles for pulmonary drug delivery,” Science, 276:1868-1871 (1997)).

Endocytosis inhibitors are also suitable macrophage inhibiting agents that can be used in the methods of the invention. Such macrophage inhibiting agents include but are not limited to, agents that affect the actin cytoskeleton, microtubules and/or microfilaments (e.g., cytochalasin D, colchicine, nocodazole), agents that affect plasma membrane fluidity (e.g., benzyl alcohol, anaesthetics (e.g., procaine, dibucaine, tetracaine)), agents that sequester and/or complex with cholesterol (e.g., cyclodextrins, filipin, nystatin, progesterone) and/or cationic amphiphiles that interact with membrane (e.g., chloropromazine, dansylcadaverine, amantadine)).

Macrophage inhibiting agents also include EDTA-containing liposomes and Ca²⁺-EDTA-containing liposomes, both of which are effective for inhibiting macrophage uptake of an agent (e.g., biologically active agent) (see, e.g., Naito, M. et al., “Liposome-encapsulated dichloromethylene diphosphonate induces macrophage apoptosis in vivo and in vitro,” J. Leukoc. Biol., 60(3):337-344 (1996)). Other macrophage inhibiting agents include lysomotropic agents (e.g., chloroquine, ammonium chloride, monensin). Combinations of any of the macrophage inhibiting agents are also encompassed for use in the methods of the invention.

In one embodiment, the method uses a macrophage inhibiting agent which is not one or more of amino acids, an endocytosis inhibitor, EDTA-containing liposomes, Ca²⁺-EDTA-containing liposomes or a lysomotropic agent.

The term “biologically active agent,” as used herein, is an agent, or its pharmaceutically acceptable salt, which, when released in vivo, possesses the desired biological activity, for example therapeutic, diagnostic and/or prophylactic properties in vivo. It is understood that the term includes stabilized biologically active agents as described herein.

Examples of suitable biologically active agents include proteins such as immunoglobulins, antibodies, cytokines (e.g., lymphokines, monokines, chemokines), interleukins, interferons, erythropoietin, nucleases, tumor necrosis factor, colony stimulating factors, insulin, enzymes (e.g. superoxide dismutase, plasminogen activator, etc.), tumor suppressors, blood proteins, hormones and hormone analogs (e.g., growth hormone (e.g., mammalian growth hormone, in particular human growth hormone), adrenocorticotropic hormone, luteinizing hormone releasing hormone (LHRH), gonadotropin-releasing hormone (GHRH), leoprolide, granulocyte colony-stimulating factor (G-CSF), parathyroid hormone-related peptide, somatostatin, testosterone, progesterone, estradiol), vaccines (e.g., tumoral, bacterial and viral antigens), antigens, blood coagulation factors; growth factors; peptides such as protein inhibitors, protein antagonists, and protein agonists; nucleic acids, such as antisense molecules; oligonucleotides; and ribozymes. Small molecular weight agents (e.g., small organic molecules) suitable for use in the invention include, for example, antitumor agents such as bleomycin hydrochloride, carboplatin, methotrexate and adriamycin; antibiotics such as gentamicin, tetracycline hydrochloride and ampicillin; antipyretic, analgesic and anti-inflammatory agents; antitussives and expectorants such as ephedrine hydrochloride, methylephedrine hydrochloride, noscapine hydrochloride and codeine phosphate; sedatives such as chlorpromazine hydrochloride, prochlorperazine hydrochloride and atropine sulfate; muscle relaxants such as tubocurarine chloride; antiepileptics such as sodium phenyloin and ethosuximide; antiulcer agents such as metoclopramide; antidepressants such as clomipramine; antiallergic agents such as diphenhydramine; cardiotonics such as theophillol; antiarrhythmic agents such as propranolol hydrochloride; vasodilators such as diltiazem hydrochloride and bamethan sulfate; hypotensive diuretics such as pentolinium and ecarazine hydrochloride; antidiuretic agents such as metformin; anticoagulants such as sodium citrate and sodium heparin; hemostatic agents such as thrombin, menadione sodium bisulfite and acetomenaphthone; antituberculous agents such as isoniazide and ethanbutol; hormones such as prednisolone sodium phosphate and methimazole; antipsychotic agents such as risperidone; and narcotic antagonists such as nalorphine hydrochloride. Other specific biologically active agents include, but are not limited to, calcitonin, nicotine, fentanyl, norethisterone, clonidine, scopolamine, salicylate, cromolyn sodium, salmeterol, formeterol, ipratropium bromide, albuterol (including albuterol sulfate), fluticasone, valium, alprazolam and levodopa (L-Dopa). In addition, suitable biologically active agents include those listed in U.S. Pat. No. 5,875,776 and U.S. application Ser. No. 09/665,252, filed Sep. 19, 2000 (Atty. Docket Number 2685.1009-000), the teachings of both of which are incorporated herein by reference in their entirety. Those therapeutic agents which are charged, such as many of the peptides and proteins (e.g., insulin), can be administered as a complex between the charged agent and a molecule of opposite charge. Preferably, the molecule of opposite charge is a charged lipid or an oppositely-charged protein.

In some embodiments, the biological active agent is not an antigen or vaccine.

In a particular embodiment, the biologically active agent is selected from the group consisting of insulin, growth hormone (e.g., human growth hormone) and immunglobulins.

In certain embodiments of the invention, the biologically active agent is an agent that has a molecular weight that is equal to or greater than 5,000 Daltons. In other embodiments, the biologically active agent is an agent that is equal to or greater than 10,000 Daltons, 20,000 Daltons, 30,000 Daltons, 40,000 Daltons or 50,000 Daltons, respectively. Biologically active agents that have a molecular weight equal to or greater than these values have a higher likelihood of saturable uptake and, thus, are well suited for the methods of the invention.

In certain embodiments of the invention, the biologically active agent is selected from the group consisting of a small organic molecule, a protein, a peptide, a peptidomimetic and a nucleic acid. In other embodiments, the biologically active agent is a protein or a peptide. In still other embodiments, the biologically active agent is a protein that has a molecular weight that is equal to or greater than 10,000 Daltons, 20,000 Daltons, 30,000 Daltons or 40,000 Daltons, respectively. Proteins that have a molecular weight equal to or greater than these values have a higher likelihood of saturable uptake and, thus, are well suited for the methods of the invention.

In a particular embodiment, the biologically active agent (e.g., protein, peptide) has a rate of transport through the cell linings of a pulmonary system (K(1)) that is lower than the rate of degradation of the protein or peptide in the pulmonary system (K(2)). Biologically active agents that have a K(1) value that is lower than K(2) undergo saturable uptake, and therefore are particularly amenable to the methods of the invention.

The term “peptide,” as used herein, refers to a compound consisting of from about two to about 100 amino acid residues, wherein the amino group of one amino acid is linked to the carboxyl group of another amino acid by a peptide bond. The term “protein,” as used herein, refers to a compound consisting of 100 or more amino acid residues, wherein the amino group of one amino acid is linked to the carboxyl group of another amino acid by a peptide bond.

“Bioavailability,” as that term is used herein, refers to the amount of a biologically active agent that becomes available to target tissue after administration. The term is meant to encompass both the amount of biologically active agent that reaches the general circulation (e.g., for agents that are targeted to the general circulation), as well as the pulmonary system (e.g., for pulmonary agents that have a local biological effect in the pulmonary system). Determination of bioavailability is well known in the art and can be calculated by measuring the Area Under the Curve (AUC) for the release profile of a particular biologically active agent over a period of time. Bioavailability is often referred to in terms of % Bioavailability, which in the case of systemic absorption, is the bioavailability achieved for a particular biologically active agent (e.g., a protein, a peptide) following pulmonary administration, divided by the bioavailability achieved for the same biologically active agent administered by injection (e.g., intravenous, subcutaneous, intramuscular, intraperitoneal) multiplied by 100. Generally, the dose delivered to the pulmonary system and the dose delivered by injection are adjusted so that the AUCs are similar, which often requires a larger pulmonary dose to be delivered.

The methods of the invention are drawn to pulmonary delivery of a biologically active agent. Pulmonary delivery of biologically active agents is well known in the art and includes, but is not limited to, delivery of dry powder formulations (DPF's) (e.g., particles, nanoparticles), liquids, solutions, suspensions, crystals, liposomes (see, e.g., U.S. Pat. No. 5,855,913 (Edwards, D. A., et al.), U.S. Pat. No. 5,985,309 (Edwards, D. A., et al.), Liu, R., et al., Biotechnol. Bioeng., 37: 177-184 (1991); Damms, B., et al., Nature Biotechnology (1996); Kobayashi, S., et al., Pharm. Res., 13(1):80-83 (1996); Timsina, M. et al., Int. J. Pharm., 101: 1-13 (1994)). Methods of delivery of biologically active agents (e.g., using nebulizers, dry powder inhalers, metered dose inhalers (MDI's; e.g., pressurized MDI's), single-step or multi-step inhalers, intratracheal administration) are also well known in the art. For example, pulmonary administration of particles is taught in U.S. Pat. Nos. 5,855,913, 5,985,309 and 6,136,295, the teachings of all of which are incorporated herein by reference in their entirety.

In one embodiment, the biologically active agent and the macrophage inhibiting agent are co-administered. As used herein, co-administration refers to simultaneous administration of the biologically active agent and the macrophage inhibiting agent. The methods of the invention also encompass administration of the macrophage inhibiting agent before and/or after administration of the biologically active agent. In one embodiment, the macrophage inhibiting agent is administered prior to the biologically active agent.

In one embodiment, the biologically active agent and the macrophage inhibiting agent are present in a single composition. In another embodiment, the single composition comprising the biologically active agent and the macrophage inhibiting agent are particles. In still another embodiment, the particles have a tap density of less than about 0.4 g/cm³. Particles which have a tap density of less than about 0.4 g/cm³ are referred to herein as “aerodynamically light particles”. For example, the particles have a tap density less than about 0.3 g/cm³, or a tap density less than about 0.2 g/cm³, or a tap density less than about 0.1 g/cm³. Tap density is measured by using instruments known to those skilled in the art such as the Dual Platform Microprocessor Controlled Tap Density Tester (Vankel, N. C.) or a GeoPyc™ instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10^{th} Supplement, 4950-4951, 1999. Features which contribute to low tap density include irregular surface texture and porous structure.

In addition, particles which may be used in the invention can be of a preferred size, e.g., a volume median geometric diameter (VMGD) of at least about 1 micron (μm). In one embodiment, the particles of the invention have a VMGD is from about 1 μm to 30 μm, or any subrange encompassed by about 1 μm to 30 μm, for example, but not limited to, from about 5 μm to about 30 μm. For example, the particles have a VMGD ranging from about 1 μm to 10 μm, or from about 3 μm to 7 μm, or from about 5 μm to 15 μm. The particles can also have a median diameter, mass median diameter (MMD), a mass median envelope diameter (MMED) or a mass median geometric diameter (MMGD) of at least 1 μm, for example, 5 μm or near to or greater than about 10 μm. For example, the particles have a MMGD greater than about 1 μm and ranging to about 30 μm, or any subrange encompassed by about 1 μm to 30 μm, for example, but not limited to, from about 5 μm to 30 μm or from about 10 μm to about 30 μm.

Particles that can be used in the invention can also have a “mass median aerodynamic diameter” (MMAD), also referred to herein as “aerodynamic diameter,” between about 1 μm and about 5 μm or any subrange encompassed between about 1 μm and about 5 μm. For example, such particles can have, but are not limited to, a MMAD that is between about 1 μm and about 3 μm, or a MMAD that is between about 3 μm and about 5 μm.

Experimentally, aerodynamic diameter can be measured using an AeroDisperser/Aerosizer. The sample powder was aerosolized by an inlet air stream at 1 psi in the AeroDisperser and then accelerated to sonic velocity into the Aerosizer. The Aerosizer measures the time taken for each particle to pass between two fixed laser beams, which is dependent on the particle's inertia. The time of flight (TOF) measurements were subsequently converted into aerodynamic diameters using Stokes law. Additionally, the aerodynamic diameter can be determined by employing a gravitational settling method, whereby the time for an ensemble of particles to settle a certain distance is used to infer directly the aerodynamic diameter of the particles. The MSLI also provides an indirect method for measuring the mass median aerodynamic diameter.

The aerodynamic diameter, dₐₑᵣ, can be calculated from the equation:
dₐₑᵣ=d_{g}√ρ tap
where d_{g} is the geometric diameter, for example the MMGD and ρ is the powder density.

Particles which have a tap density less than about 0.4 g/cm³, median diameters of at least about 1 μm, for example, at least about 5 μm, and an aerodynamic diameter of between about 1 μm and about 5 μm, preferably between about 1 μm and about 3 μm, are more capable of escaping inertial and gravitational deposition in the oropharyngeal region, and are targeted to the airways or the deep lung. In addition, the use of larger, more porous particles is advantageous since they are able to aerosolize more efficiently than smaller, denser aerosol particles such as those currently used for inhalation therapies.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 μm are preferred for delivery to the central and upper airways. Particles having an aerodynamic diameter ranging from about 1 to about 3 μm are preferred for delivery to the deep lung. Thus, for particulate compositions that comprise a biologically active agent and/or a macrophage inhibiting agent, the particulate composition can be manufactured for optimal delivery to a selected region of the respiratory tract (e.g., deep lung, upper or central airways).

“Subject” as that term is used herein refers to the recipient of the methods of the invention. Mammalian and non-mammalian subjects are included. In a specific embodiment, the subject is a mammal, such as a human, canine, murine, feline, bovine, ovine, swine or caprine. In a preferred embodiment, the subject is a human.

The term “sustained release composition” as defined herein, refers to a composition which comprises a biologically active agent, wherein the composition comprising the biologically active agent has a duration of biological activity that is greater than that of the biologically active agent by itself. Sustained release compositions are well known in the art and include, e.g., biodegradable particles with controlled release properties (see, e.g., Langer, R., Science, 249:1527-1533 (1990); the entire teachings of which are incorporated herein by reference), polymeric compositions (see, e.g., U.S. patent application Ser. No. 09/835,001, filed on Apr. 13, 2001 and entitled “Method of Modifying the Release Profile of Sustained Release Compositions”; the entire teachings of which are incorporated herein by reference), incorporation of surfactants (e.g., lipids) (see, e.g., U.S. Pat. No. 5,855,913). In certain embodiments, the methods of the invention administer a sustained release composition which comprises a biologically active agent.

In another aspect, the invention is a method of enhancing pulmonary absorption of a biologically active agent comprising administering to a pulmonary system of a subject a biologically active agent and a macrophage inhibiting agent that is suitable for administration to a pulmonary system. Suitable macrophage inhibiting agents include, e.g., those described herein.

In order to optimize delivery of a biologically active agent to, and uptake by, a subject's pulmonary system, it is important to consider various rate constants. The first rate constant which is important to consider is the rate of release of agent from delivery vehicle (defined herein as K(0)). The rate of release of a biologically active agent from a delivery vehicle depends on a number of factors, including the type of agent, the formulation of the agent, the manner of administration (e.g., dry powder, crystals, liposomes, liquid), the presence of excipients, the presence of complexing agents that form complexes with the agent and alter its release (e.g., delaying release), and the like. A high K(0) means that the agent is rapidly presented to the lung (fast delivery), while a low K(0) means that the agent is slowly presented to the lung (e.g., a formulation that promotes sustained delivery).

Although K(0) can be calculated or measured in a variety of ways, as defined herein, K(0) refers to the rate of release of agent from delivery vehicle as calculated in the following manner. The rate of release of agent from delivery vehicle (K(0)) can be described in terms of release constants. The first order release constant can be expressed using the following equations:
M₍ₜ₎=M₍ₒₒ₎*(1−e^{-k*t}) (Equation 1)
where k is the first order release constant, M₍ₒₒ₎ is the total mass of agent in the agent delivery vehicle (e.g. a dry powder formulation) and M₍ₜ₎ is the mass of agent released from the delivery vehicle at time t.

Equation 1 may be expressed either in amount (i.e., mass) of agent released or concentration of agent released in a specified volume of release medium. For example, Equation 1 may be expressed as:
C₍ₜ₎=C₍ₒₒ₎*(1−e^{-k*t}) or Release₍ₜ₎=Release₍ₒₒ₎*(1−e^{-k*t}) (Equation 2)
where k is the first order release constant, C₍ₒₒ₎ is the maximum theoretical concentration of agent in the release medium, and C₍ₜ₎ is the concentration of agent being released from the delivery vehicle to the release medium at time t.

Agent release rates in terms of first order release constants can be calculated using the following equation:
k=−ln(M₍ₒₒ₎−M₍ₜ₎)/M₍ₒₒ₎/t (Equation 3)
where k is the first order release constant, M₍ₒₒ₎ is the total mass of agent in the agent delivery vehicle (e.g. a dry powder formulation) and M₍ₜ₎ is the mass of agent released from the delivery vehicle at time t. Using this equation, one can calculate k (i.e., K(0); the rate of release of agent from delivery vehicle).

A second rate constant that is important for optimizing delivery of a biologically active agent to the lungs is the rate of transport through the cell linings of the lung (defined herein as K(1)). Although K(1) can be calculated using a variety of in vitro, in vivo and ex vivo assays (see, e.g., Kim, K. J., et al., Pharm. Res., 18(3):253-255 (2001); Kim, K. J., et al., Am. J. Physiol. Lung Cell Mol. Physiol., 284(2):L247-L259 (2003); Eljamal, M., et al., “In situ and in vivo methods for pulmonary delivery,” In Bordcharth, R. T., Smith, P. L., and Wilson, G., (Eds.), Models for Assessing Drug Absorption and Metabolism, (New York, Plenum Press, 1996), pp. 361-374; Komada, F., et al., J. Pharm. Sci., 83:863-867 (1994); Patton, J. S., et al., J. Control. Rel., 28:79-85 (1994); Tronde, A., et al., Peptides, 23:469-478 (2002); the teachings of all of which are incorporated herein by reference in their entirety), as defined herein, K(1) refers to the rate of transport through the cell linings of the lung as measured using the in vitro assay described by Kim et al. (Kim, K.-J., et al. “Absorption of intact albumin across rat alveolar epithelial cell monolayers,” Am. J. Physiol. Lung Cell Mol. Physiol., 284:L458-L465 (2003); the entire teachings of which are incorporated herein by reference). This assay utilizes an in vitro model of primary cultured rat pneumocyte monolayers grown on tissue culture-treated polycarbonate filters to measure K(1). These monolayers comprise alveolar epithelial type I-like cells, develop high barrier resistance and actively reabsorb Na⁺ from apical fluid (Id., at L458). Using this assay, Kim et al. were able to show that fluorescein isothiocyanate (FITC)-labeled bovine serum albumin (F-Alb) transports across the alveolar epithelial barrier predominately via transcellular saturable processes (e.g., transcytosis) mediated by specific albumin-binding sites (Id., at L459). Specifically, Kim et al. demonstrated that net absorption of intact F-Alb exhibited a simple saturable process with Kₜ (i.e., K(1))≠1.6 μM and a Jₘₐₓ (i.e., maximum flux)˜0.15 fmol.cm⁻².s⁻¹. For many biologically active agents, and in particular biologically active agents of relatively large molecular weight (e.g., biologically active agents greater than 40,000 Daltons), transport through the cell linings of the lung is a saturable process. In general, transport through the cell linings of the lung into the bloodstream of a subject appears to be roughly proportional to the molecular weight of the transporting molecule.

A third rate constant that is important for optimizing delivery of a biologically active agent to the lungs is the rate of degradation of the agent in the lungs (defined herein as K(2)). Chemical or physical degradation of a biologically active agent in the lung occurs via various degradation pathways, including degradation by alveolar macrophages. As demonstrated herein, macrophage inhibitors (e.g., bisphosphonate compounds (e.g., dichloromethylene diphosphonate)) reduce K(2) and thereby increase pulmonary absorption and bioavailability. Although it is possible to measure degradation by alveolar macrophages using a variety of in vitro and in vivo assays (e.g., Kooistra, T., et al., Biosci. Rep., 1(7):587-594 (1981); Besterman, J. M., et al., J. Cell Biol., 96(6):1586-1591 (1983); Ehrenreich, B. A., et al., J. Exp. Med., 126(5):941-958 (1967); Williams, K. E., et al., J. Cell Biol., 64(1):123-134 (1975); Kim, K. J., et al., Pharm. Res., 18(3):253-255 (2001); Kim, K. J., et al., Am. J. Physiol. Lung Cell Mol. Physiol., 284(2):L247-L259 (2003); the teachings of all of which are incorporated herein by reference in their entirety), as defined herein, K(2) refers to the rate of degradation of the agent in the lungs as calculated using the in vitro model of primary cultured rat pneumocyte monolayers taught by Kim et al. and described above for the determination of K(1) (see Kim, K.-J., et al., Am. J. Physiol. Lung Cell Mol. Physiol. 284:L458-L465 (2003)). Although Kim et al. do not explicitly teach the calculation of K(2), the person of ordinary skill in the art could use the in vitro model of primary cultured rat pneumocyte monolayers and isolated macrophages (e.g., suspensions of macrophages) to calculate K(2).

As demonstrated herein, in order to enhance bioavailability of an inhaled agent, it is desirable to have K(1) be greater than K(2), e.g., wherein K(1) is at least 5 times greater than K(2). Thus, in one embodiment, the invention is a method of enhancing bioavailability of a biologically active agent that is administered to the pulmonary system of a subject, comprising:
a) determining for a formulation comprising a biologically active agent:
   i) the rate of transport of said biologically active agent through the cell linings of a pulmonary system (K(1)); and
   ii) the rate of degradation of said biologically active agent in the pulmonary system (K(2)); and
b) administering a dose of said biologically active agent, wherein K(1) is at least 5 times greater than K(2).

In other embodiments, a dose of the biologically active agent is administered wherein K(1) is at least 10 times greater than K(2), at least 15 times greater than K(2), and at least 20 times greater than K(2), respectively.

In the case where transport of a biologically active agent through the cell linings of a pulmonary system is a saturable process, it is also desirable to have K(0) be less than or equal to K(1) in order to enhance bioavailability. Thus, in certain embodiments,
a) transport of said biologically active agent through the cell linings of a pulmonary system is a saturable process, and K(1) is the rate of transport of said biologically active agent through the cell linings of a pulmonary system at saturation; and
b) said method further comprises:
   i) determining the rate of release of said biologically active agent from a delivery vehicle (K(0)); and
   ii)) administering a dose of said biologically active agent wherein K(0) is less than or equal to K(1).

In another embodiment, K(0) is minimized by administering a sustained release composition (e.g., a sustained release composition as taught herein) that comprises the biologically active agent. The utilization of a sustained release composition allows the biologically active agent to be released at a rate that minimizes the availability of the agent to degradation (e.g., degradation by alveolar macrophages). In addition, delivery of the biologically active agent by more frequent, lower dose inhalations can also help lower K(0).

As demonstrated herein, enhancement of bioavailability of an inhaled agent occurs when K(1) be greater than K(2) (e.g., wherein K(1) is at least 5 times greater than K(2)). Thus, in certain embodiments of the invention, K(2) is reduced by administering a macrophage inhibiting agent suitable for administration to a pulmonary system. Suitable macrophage inhibiting agents include, e.g., macrophage inhibiting agents described herein (e.g., bisphosphonate compound (e.g., dichloromethylene diphosphonate)).

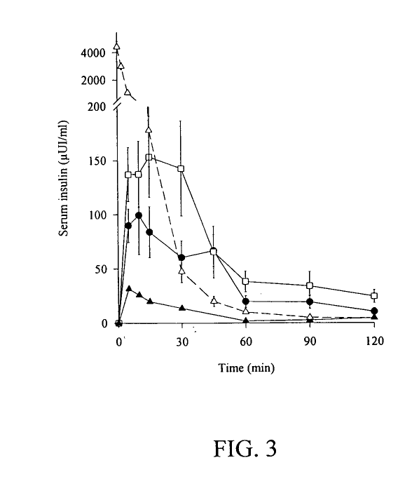

In other embodiments, K(1) is increased by administering one or more phospholipids, surfactants and/or excipients to the pulmonary system of said subject; and/or administering phosphate buffered saline to the pulmonary system of said subject. As demonstrated herein, administration of PBS liposomes prior to intratracheal instillation of insulin resulted in an increase in bioavailability, as compared to untreated rats administered only insulin (see FIG. 3).

In other embodiments, enhancement of bioavailability is achieved by modifying K(0), K(1) and K(2). For example, in one embodiment,
a) K(2) is reduced by administering a macrophage inhibiting agent suitable for administration to a pulmonary system;
b) K(0) is minimized by administering a sustained release composition comprising said biologically active agent; and
c) K(1) is increased by:
   i) administering one or more phospholipids, surfactants and/or excipients to the pulmonary system of said subject; and/or
   ii) administering phosphate buffered saline to the pulmonary system of said subject.

In another aspect, the invention is a method of determining an optimal dosing regimen for pulmonary delivery of a biologically active agent comprising:
a) determining K(1) and K(2); and
b) selecting a dosing regimen of said biologically active agent, wherein K(1) is at least 5 times greater than K(2).
   In another embodiment, the method of determining an optimal dosing regimen further comprises determining K(0) and administering a dose of said biologically active agent wherein K(0) is less than or equal to K(1).

In still another aspect, the invention is a method of conducting and optimizing dosing for pulmonary delivery of a biologically active agent comprising:
a) determining K(1) and K(2); and
b) selecting a dosing regimen of said biologically active agent, wherein K(1) is at least 5 times greater than K(2).
   In another embodiment, the method of conducting and optimizing dosing further comprises determining K(0) and administering a dose of said biologically active agent wherein K(0) is less than or equal to K(1). In still another embodiment, the method further comprises formulating a pharmaceutical preparation comprising the biologically active agent at a dose which comports with the selected dosing regimen.

In another aspect, the invention is a method of administering a therapeutically active agent to the pulmonary system of a subject comprising administering a therapeutically active agent and a macrophage inhibiting agent suitable for administration to a pulmonary system. As used herein, a therapeutically active agent includes biological agents that have a therapeutic, prophylactic and/or diagnostic effect when administered in vivo. Such therapeutically active agents include, for example, the biologically active agent described herein, but does not include antigenic compounds that do not possess a therapeutic, prophylactic and/or diagnostic effect (e.g., antigenic agents utilized to study pulmonary immunity). In a preferred embodiment, the method comprises administering a therapeutically active agent selected from the group consisting of an immunoglobulin, insulin and growth hormone. Macrophage inhibiting agents include those described herein. In a preferred embodiment, the macrophage inhibiting agents is a bisphosphonate compound (e.g., dichloromethylene diphosphonate, alendronate, risendronate, pamidronate, etidronate, tiludronate). In another embodiment, the macrophage inhibiting agent is present in an amount sufficient to enhance bioavailability of said biologically active agent.

### EXAMPLES

### Example 1

This Example demonstrates that a primary source of elimination of inhaled biologically active agents (e.g., macromolecules) following delivery to the lungs and prior to absorption into the systemic circulation is a result of clearance by alveolar macrophages (AM). Depletion of alveolar macrophages by a macrophage inhibiting agent (e.g., a bisphosphonate compound (e.g., liposome-encapsulated dichloromethylene diphosphonate)) results in a several-fold enhancement in systemic absorption and bioavailability of biologically active agents (e.g., protein, (e.g., immunoglobulin G (IgG), human chorionic gonadotrophin (hCG)) following intratracheal instillation in rats.

This Example also demonstrates that decreasing the dose of biologically active agent (e.g., protein, (e.g., IgG)) that is delivered to the lungs alleviates local degradation and results in a relative increase in systemic absorption and bioavailability of the agent as well.

Experimental Protocol

Confocal Laser Scanning Microscopy.

Rat immunoglobulin G1 anti-dinitrophenyl hapten (IgG, LO-IMEX, Brussels, Belgium) was custom labeled with fluorescein isothiocyanate (FITC) and the labeled protein localized by confocal laser scanning microscopy (CLSM) in the pulmonary tissue following intratracheal instillation in rats, as previously described (Lombry C., et al., “Confocal imaging of rat lungs following intratracheal delivery of dry powders or solutions of fluorescent probes,” J. Control. Release, 83:331-341 (2002)). Briefly, 12-week old male Wistar rats (Elevage Janvier, Le Genest St Isle, France) were anesthetized with ketamine/xylazine (90/10 mg/kg) intraperitoneal injection and 500 μg FITC-IgG dissolved in 100 μl NaCl 0.9% was instilled in the lungs using a Microsprayer™ device (PennCentury, Inc., Philadelphia, Pa.) inserted in the trachea via the mouth. Within 1 minute and at intervals up to 4 days after intratracheal delivery, the lungs were lavaged and fixed by vascular perfusion of phosphate buffered saline (PBS; pH 7.4) containing sulforhodamine (0.1%; Sigma-Aldrich, Bornem, Belgium) for 5 minutes followed by a fixative solution (formaldehyde 0.6%, glutaraldehyde 0.9%, Na cacodylate 75 mM, sulforhodamine 0.1%, adjusted to pH 7.4) for an additional 5 minutes. The thoracic cavity was opened and the lungs were removed for analysis by CLSM (BioRad MRC 1024 confocal unit). Each experimental condition was repeated at least 3 times.

Slices (±2 mm) of the lobes and trachea were placed directly in a sample holder and covered with a coverslip glass. Laser excitation wavelengths of 488 nm and 568 nm were used individually to scan lung tissue and fluorescent emissions from FITC-IgG (emission λ=515-545 nm) and sulforhodamine (emission λ=589-621 nm) were collected using separate channels. Images were acquired with a Zeiss Plan-Neofluor 40×oil immersion lens and 10×lens.

The uptake of FITC-IgG by AM was further visualized by analyzing AM collected by broncho-alveolar lavage (BAL). BAL was carried out as previously described (Lombry C., et al., J. Control. Release, 83:331-341 (2002)) and a few droplets of the cells suspension were placed directly in a sample holder and covered with a coverslip glass for analysis by CLSM.

Preparation of Dichloromethylene Diphosphonate (CL₂MDP) and PBS Liposomes.

86 mg of phosphatidylcholine (Lipoid GMBH, Ludwigshafen, Germany) and 10 mg of cholesterol (Sigma-Aldrich, Bornem, Belgium) were dissolved in 10 ml of chloroform. A lipid film was produced by low vacuum rotary evaporation and the film was dispersed by gentle rotation in 10 ml of PBS or 10 ml of dichloromethylene diphosphonate (Cl₂MDP; Sigma-Aldrich) solution (2.5 g/10 ml sterile water). After complete removal of the film, the suspension was left for 30 minutes at room temperature under nitrogen atmosphere. The suspension was then sonicated for 2 minutes at a power of 60 W (Sympatec GmbH, Clausthal-Zellerfelg, Germany). Free Cl₂MDP was removed by dilution of the suspension in 100 ml of PBS and centrifugation at 10,000 rpm for 30 minutes. The liposomes pellet was resuspended in 1.5 ml of PBS. The suspension of Cl₂MDP or PBS liposomes was stored under nitrogen atmosphere at 4° C. for a maximum of one week (see also, Van Rooijen N., et al., “Liposome mediated depletion of macrophages: mechanism of action, preparation of liposomes and applications,” J. Immunol. Methods, 174:83-93 (1994)).

Depletion or Intratracheal Instillation of AM.

750 μl of suspension of Cl₂MDP liposomes or PBS liposomes was instilled in the trachea of anesthetized rats using a syringe inserted via the mouth. AM depletion resulting from Cl₂MDP was assessed by analyzing cellular components in BAL at 1, 2.5 and 5 days after delivery of the liposomes. The total number of cells was determined by mixing with Turch liquid and counting with a hemacytometer. AM were differentiated from other cells on cytocentrifuge preparations fixed in methanol and stained with Diff-Quik (300 cells/rat; Dade Berhing AG, Düdingen, Switzerland). No structural alterations to airway or alveolar epithelia were visible in hematoxylin/eosin-stained histological sections 1 day after intratracheal instillation of either PBS or Cl₂MDP liposomes.

An increase in the number of AM that were present locally in the lungs was achieved by instilling additional AM into the lungs of intact rats. AM were collected by BAL, and the suspensions of AM from several rats were pooled. 5 million cells (a BAL from a single rat yields an average of 2.5 million AM) were suspended in 200 μl of Hank's balanced salt solution (Life Technologies, Merelbeke, Belgium) and the cell-containing solution was intratracheally instilled into the lungs of each intact animal.

Pharmacokinetic Studies.

Rats (12 week old male Wistar, 413±21 g; Elevage Janvier) were intratracheally instilled with liposomes or additional AM 1 day or 1 h, respectively, before pulmonary administration of the therapeutic protein or peptide. In addition, control rats remained untreated. The rats were anesthetized using ketamine/xylazine intraperitoneal injection and received either 5, 50 or 500 μg of IgG, 100 μg of hCG (Profasi®, Serono Benelux, Brussels, Belgium) or 40 μg of human insulin (ICN Biomedicals, Ohio, USA) by intratracheal instillation (100 μl NaCl, 0.9% solution). In addition, other rats received 5 or 50 μg of IgG, 10 μg of hCG or 10 μg of human insulin by intravenous injection (500 μl NaCl 0.9% solution).

The 3 doses of IgG that were delivered to the lungs were selected according to the quantities of total IgG that is naturally present in the lungs of untreated rats (52±13 μg, as measured in BAL). Thus, the dosage range that was selected included a low (5 μg), an average (50 μg) and a high (500 μg) dose, as compared to the quantity of IgG that is naturally present in the lungs of untreated rats. Serum was collected by orbital bleeding at intervals up to 42 days, 3 days, or 6 h after administration of IgG, hCG or insulin, respectively. All experimental protocols in rats were approved by the Institutional Animal Care and Use Committee of the University Catholique de Louvain. IgG concentrations in serum were measured by enzyme-linked immunosorbent assay (ELISA). Plates (Nunc-Immuno Plate Maxisorp Surface, Gibco BRL Life Technologies) were coated with dinitrophenyl human albumin (Sigma-Aldrich), incubated with dilutions of sera, and developed with horseradish peroxidase-conjugated anti-rat IgG Fc region (Pharmingen, Becton Dickinson Biosciences, Erembogem, Belgium). Serum levels of hCG and insulin were measured by enzyme immunoassay (Biosource, Nivelles, Belgium) and immunoradiometric assay (Biosource, Nivelles, Belgium), respectively.

The areas under the serum concentration-time curves (AUC) were calculated using the linear trapezoidal rule and the absolute bioavailability values of the intratracheal instillations (IT) were calculated by comparison to intravenous injection (IV) as

$\frac{{AUC}_{IT} * {Dose}_{IV}}{{AUC}_{IV} * {Dose}_{IT}} * 100$

Statistics.

The data were validated using the Dixon test. Results are expressed as mean values±standard error of the mean (SEM). A one-way analysis of variance (ANOVA) test and the Tukey test were performed to demonstrate statistical differences (p<0.05), using the software Sigma-stat for Windows (SPSS Inc., San Rafael, Calif., USA).

Results and Discussion

Fate of Immunoglobulins in the Lungs.

In order to assess visually the fate of a large macromolecule in the lungs, we delivered rat immunoglobulin G (IgG) to the lungs of rats and examined protein fate in pulmonary tissue post-delivery using confocal laser scanning microscopy. FITC-labeled IgG slowly disappeared from the pulmonary tissue over a period of 24 h after intratracheal spray-instillation. An intense uptake of IgG (which primarily appeared in soluble form, although some particulate IgG was also visible by AM) arose as early as 10 minutes following delivery and remained prominently visible for up to 3 days. This finding coincides with previous published observations of AM uptake for different size macromolecules (Hastings R. H., et al., “Cellular uptake of albumin from lungs of anesthetized rabbits,” Am. J. Physiol., 269: L453-L462 (1995); Welty-Wolf, K. E., et al., “Aerosolized manganese SOD decreases hyperoxic pulmonary injury in primates. II. Morphometric analysis,” J. Appl. Physiol., 83:559-568 (1997); Lombry, C., et al., “Confocal imaging of rat lungs following intratracheal delivery of dry powders or solutions of fluorescent probes,” J. Control. Release, 83:331-341 (2002)).

Effect of AM Uptake on Systemic Bioavailability of IgG.

Previous studies have either not assessed (e.g., Hastings R. H., et al., Am. J. Physiol., 269:L453-L462 (1995); Welty-Wolf, K. E., et al., J. Appl. Physiol., 83:559-568 (1997); Lombry C., et al., J. Control. Release, 83:331-341 (2002)) or have concluded a negligible effect (e.g., Berthiaume, Y., et al., “Protein clearance from the air spaces and lungs of unanesthetized sheep over 144 h,” J. Appl. Physiol., 67:1887-1897 (1989)) of protein uptake by AM, on systemic bioavailability. To test this, we eliminated AM using liposomes containing dichloromethylene diphosphonate (Cl₂MDP) and compared serum IgG levels following pulmonary administration of IgG to AM-depleted rats and control rats. Selective depletion of splenic, liver or alveolar macrophages by Cl₂MDP liposomes has previously been utilized to study macrophage function in physiology, pathology or immunity (see, e.g., Pinto, A. J., et al., “Selective depletion of liver and splenic macrophages using liposomes encapsulating the drug dichloromethylene diphosphonate: Effects on antimicrobial resistance,” J. Leuk. Bio., 49:579-586 (1991); Berg, J. T., et al., “Depletion of alveolar macrophages by liposome-encapsulated dichloromethylene diphosphonate,” J. Appl. Physiol., 74:2812-2819 (1993)).

These experiments demonstrated that a single intratracheal dose of Cl₂MDP liposomes administered to rats decreased AM number in broncho-alveolar lavage from 2.3 million to 0.5 million 1 day after delivery. The number of AM remained low for the next 36 hours and repopulation of the lungs with AM was not observed until 5 days post-administration. In contrast, animals treated with liposomes prepared with phosphate buffer saline (PBS) and no Cl₂MDP showed normal AM population throughout the examined period.

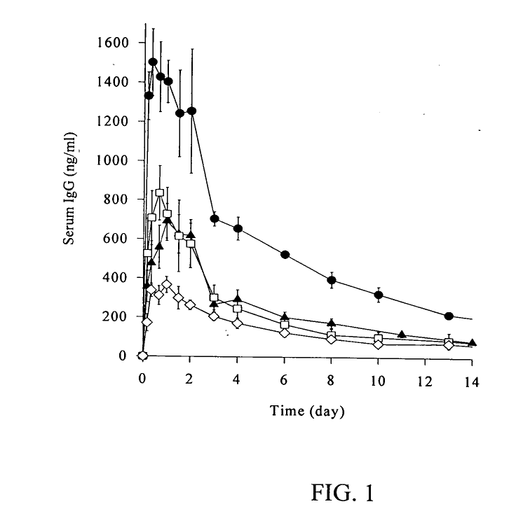

As depicted in FIG. 1, a substantial rise in serum IgG levels resulted from the depletion of AM. In the experiments depicted in FIG. 1, rats were either (i) depleted of AM using dichloromethylene diphosphonate (Cl₂MDP) liposomes (●,n=4); (ii) intratracheally administered phosphate buffer saline (PBS) liposomes (□, n=4); (iii) administered additional AM (⋄, n=5); or (iv) were left untreated (▴, n=6). One day after treatment with Cl₂MDP or PBS liposomes or 1 hour after administration of AM or no treatment, rats received an IgG dose of 500:g by intratracheal instillation.

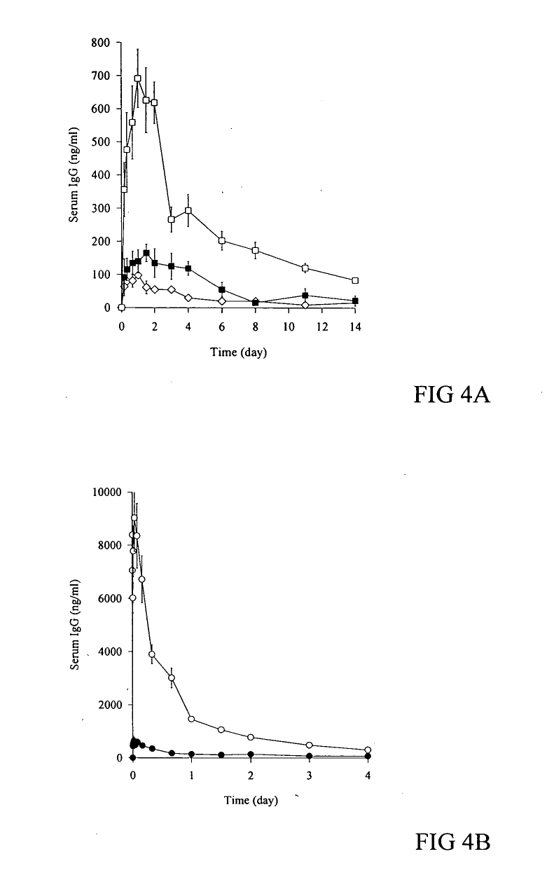

As can be seen in FIG. 1, the pulmonary bioavailability (relative to an intravenous dose of 50 μg of IgG (presented in FIG. 4b)) increased from 4.7±1.1% in untreated rats (or 4.5±2.5% in PBS liposomes-treated rats) to 10.5±3.5% in Cl₂MDP liposomes-treated rats (FIG. 1, p<0.05). Conversely, when additional AM were instilled into rat lungs, the pulmonary bioavailability of IgG decreased to 2.6±1.0% (FIG. 1, p>0.05). Values are averages±standard error of the mean (SEM). In all cases, serum IgG levels were affected by AM number within the first hours of the absorption phase (FIG. 1), indicating that AM clearance of IgG occurred rapidly. This relationship between IgG absorption from the lungs and the number of AM demonstrates that AM present a significant hindrance to IgG transport from airway lumen to the bloodstream. This is in contrast to the teachings of other references (Folkesson, H. G., et al., J. Appl. Physiol., 80:1431-1445 (1996); Wolff, R. K., J. Aerosol Med., 11:197-219 (1998); Hastings, R. H., et al., Am. J. Physiol., 269:L453-L462 (1995)).

Effect of AM Uptake on Systemic Bioavailability of hCG and Insulin.

In order to further assess the significance of AM in limiting the systemic absorption of macromolecules from the lungs, we studied the transport of human chorionic gonadotrophin (hCG, 40 kDa) from air spaces into the blood in AM-depleted and control rats. hCG was selected because of its relatively low molecular weight and because AM do not have any known receptors for hCG (Goodman Gilman, A., et al., Goodman & Gilman's—The Pharmacological Basis of Therapeutics, 8^{th} edition, McGraw Hill, Singapore (1992)). This is in contrast to IgG, which is recognized by Fc receptors on phagocytes, which have high avidity for antibodies bound to antigen but relatively low affinity for free immunoglobulin molecules (Janeway C. A., et al., Immunobiology: The immune system in health and disease, 4^{th} edition, Current Biology Publications, Elsevier, London & Garland Publishing, New York (1999)).

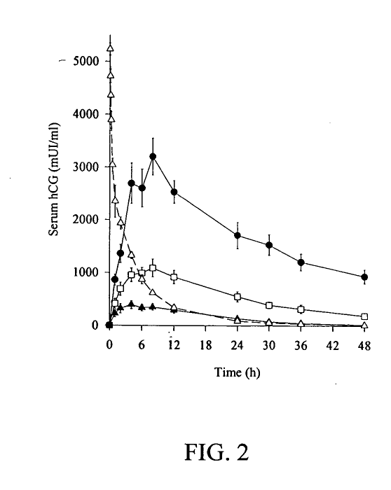

As compared to rats whose lungs remained untreated, the serum hCG levels that followed pulmonary administration of a solution of hCG were increased 3 times in rats treated with PBS liposomes and 8 times in rats treated with Cl₂MDP liposomes (FIG. 2, p<0.05). In the experiments depicted in FIG. 2, rats were left untreated (▴, n=6), administered Cl₂MDP liposomes (●, n=4) or administered PBS liposomes (□, n=6) and 1 day later were administered 100 μg of hCG by intratracheal instillation. This correlated to an enhancement in absolute bioavailability of pulmonary hCG from 4.4±1.1% (for untreated rats) to 17.6±3.7% (for rats administered PBS liposomes) to 59.7±9.2% (for rats administered Cl₂MDP liposomes), as compared to an intravenous dose of 10 μg of hCG (Δ, n=4). Values are averages±SEM. Thus, the enhancement observed in hCG transport due to AM depletion was larger than that observed for IgG transport (compare FIGS. 1 and 2). This indicates that AM were a greater barrier to hCG absorption than IgG absorption, and hence that other mechanisms (e.g., mucociliary clearance) may have been responsible for the elimination of IgG from the lungs. The influence of AM on both hCG and IgG transport suggests that AM uptake predominantly involves adsorptive and/or fluid-phase endocytosis, rather than receptor-mediated endocytosis (Mukherjee, S., et al., “Endocytosis,” Phys. Rev., 77:759-803 (1997); Kooistra, T., et al., “Serum-dependence of fluid-phase pinocytosis and specificity in adsorptive pinocytosis of simple proteins in rat peritoneal macrophages,” Biosc. Rep., 1:587-594 (1981)). Similar to the effects observed with IgG absorption, depletion of AM resulted in increased absorption of hCG into the blood early in the absorption phase (within 1 hour) (FIG. 2).

The uptake of fluid by endocytosis is a natural characteristic of professional phagocytes (Mukherjee, S., et al., Phys. Rev., 77:759-803 (1997)). Given that uptake can occur at a relatively low constant rate, significant internalization of tracers can take periods of several minutes to hours (see, e.g., Kooistra T., et al., Biosc. Rep., 1:587-594 (1981)). Thus, it was expected AM clearance would have a greater impact on pulmonary absorption of macromolecules, which can persist in the airway lumen for hours, then pulmonary absorption of smaller peptides, which may be cleared within minutes.

To test this, the impact of AM on pulmonary absorption of the peptide, insulin, was assessed. In the experiments described in FIG. 3, rats were left untreated (▴, n=6), administered Cl₂MDP liposomes (●, n=7) or administered PBS liposomes (□, n=7) and 1 day later were administered 40 μg of insulin by intratracheal instillation. The bioavailability of insulin increased from 3.1±0.7% (for untreated rats) to 13.3±3.4% (for rats administered PBS liposomes) and 8.7±2.9% (for rats administered Cl₂MDP liposomes), as compared to an intravenous dose of 10 μg (Δ, n=6). Values are averages±SEM. Thus, transport of instilled insulin to the bloodstream increased after lung treatment with PBS liposomes (p<0.05) (FIG. 3). However, in contrast to IgG and hCG, no additional increase was associated with AM depletion due to Cl₂MDP (FIG. 3, p>0.05).

Strategies for Enhancing Pulmonary Bioavailability.

These studies indicate that pinocytic uptake by AM results in the degradation of inhaled biologically active agents (e.g., macromolecules, (e.g., proteins)) thereby lowering the pulmonary bioavailability of the biologically active agent. This degradation competes with absorption of the biologically active agent across lung epithelia and thereby lowers pulmonary bioavailability so that the rate of AM uptake and degradation is near to or greater than the rate of transport from the lung lumen into the bloodstream.

Based on the enhancement in pulmonary absorption of hCG and insulin that followed pre-treatment of the lungs with PBS liposomes (FIGS. 2 and 3), it is possible to increase the rate of protein transport across the alveolar epithelium using chemical means (see, e.g., Kobayashi, S., et al., “Study on pulmonary delivery of salmon calcitonin in rats: Effects of protease inhibitors and absorption enhancers,” Pharm. Res., 11:1239-1243 (1994); Kobayashi, S., et al., “Critical factors on pulmonary absorption of peptides and proteins (diffusional barrier and metabolic barrier),” Eur. J. Pharm. Sc., 4:637-372 (1996); Li, Y., et al., “Effects of phospholipid chain length, concentration charge and vesicle size on pulmonary insulin absorption,” Pharm. Res., 13:76-79 (1996)). In this regard, co-administration of a therapeutic agent and a simple physical dispersion in saline of dipalmitoylphosphatidylcholine, the most abundant phospholipid component of pulmonary surfactant, has been reported to accelerate and increase absorption of the therapeutic agent from the lungs in rats (Mitra, R., et al., “Enhanced pulmonary delivery of insulin by lung lavage fluid and phospholipids,” Int. J. Pharm., 217:25-31 (2001)).

In addition, as demonstrated in the above-described experiments involving Cl₂MDP liposomes, reduction of the rate of uptake by AM can also increase pulmonary bioavailability. For proteins which are absorbed in a saturable manner (e.g., IgG (see, e.g., Matsukawa, Y., et al., “Rates of protein transport across rat alveolar epithelial cell monolayers,” J. Drug Target., 7:335-342 (2000); Fandy, T. E., et al., “Mechanisms of immunoglobulin G (IgG) transport across primary cultured rat alveolar epithelial cell monolayers,’ Intern. Symp. Control. Rel. Bioact. Mater., 28 (San Diego, USA; June 2001)), a decrease in the dose of the protein delivered to the lungs should favor absorption to the systemic circulation, relative to local degradation.

To test this, the experiments depicted in FIG. 4 were undertaken. In FIG. 4A, rats received a dose of 500 μg (□, n=6), 50 μg (▪, n=3) or 5 μg (⋄, n=3) IgG by intratracheal instillation. In FIG. 4B, rats received a dose of 50 μg (◯, n=4) or 5 μg (●, n=4) of IgG by intravenous injection. The 500, 50 and 5 μg doses that were administered by intratracheal instillation corresponded to an absolute bioavailability of 4.7±1.1%, 10.9±2.6% and 38.4±9.6%, respectively. Values are averages±SEM. Thus, as depicted in FIGS. 4A and 4B, a less than proportional decrease in serum IgG levels was observed for intratracheal instillation of 500 μg, 50 μg and 5 μg of IgG (FIG. 4A; i.e., an increase in absolute IgG bioavailability from 4.7% to 10.9% to 38.4% for the 500, 50 and 5 μg doses, respectively), while a proportional decrease in serum IgG levels was observed for intravenous injection of 50 μg and 5 μg of IgG (FIG. 4B).

Thus, the studies described herein demonstrate that alveolar macrophages comprise a major ‘barrier’ to transport of macromolecules from the lungs into the bloodstream, particularly for moderate to large proteins. Therefore, enhancement of the pulmonary absorption and bioavailability of a biologically active agent can be achieved by reducing AM uptake of the agent by chemical or physical means. In addition, these studies demonstrate that a reduction in the dose of agent delivered (e.g., macromolecules, protein agent (e.g., immunoglobulins)) can also provide a simple and efficient physical method to reduce degradation of the agent and increase systemic absorption of the agent from the lungs.

While this invention has been particularly shown and described with references to preferred embodiments thereof, it will be understood by those skilled in the art that various changes in form and details may be made therein without departing from the scope of the invention encompassed by the appended claims.

1. A method of enhancing bioavailability of a biologically active agent comprising administering to the pulmonary system of a subject:
a) a biologically active agent; and
b) a macrophage inhibiting agent suitable for administration to a pulmonary system,
wherein said macrophage inhibiting agent is present in an amount sufficient to enhance bioavailability of said biologically active agent.

2. The method of claim 1, wherein said macrophage inhibiting agent is administered to the pulmonary system of the subject prior to administration of said biologically active agent.

3. The method of claim 1, wherein said biologically active agent and said macrophage inhibiting agent are co-administered.

4. The method of claim 3, wherein said biologically active agent and said macrophage inhibiting agent are present in a single composition.

5. The method of claim 4, wherein said composition comprises particles.

6. The method of claim 5, wherein said particles have a tap density less than about 0.4 g/cm³.

7. The method of claim 5, wherein said particles have a volume median geometric diameter of from about 5 micrometers to about 30 micrometers.

8. The method of claim 5, wherein said particles have an aerodynamic diameter of from about 1 micrometer to about 5 micrometers.

9. The method of claim 5, wherein said particles have a tap density less than about 0.4 g/cm³, a volume median geometric diameter of about 5 micrometers to about 30 micrometers, and an aerodynamic diameter of from about 1 micrometer to about 5 micrometers.

10. The method of claim 1, wherein said macrophage inhibiting agent is a bisphosphonate compound.

11. The method of claim 10, wherein said bisphosphonate compound is selected from the group consisting of dichloromethylene diphosphonate, alendronate, risendronate, pamidronate, etidronate, tiludronate and a combination thereof.

12. The method of claim 11, wherein said bisphosphonate compound is dichloromethylene diphosphonate.

13. The method of claim 1, wherein said macrophage inhibiting agent is administered as liposomes.

14. The method of claim 1, wherein said macrophage inhibiting agent is selected from the group consisting of EDTA-containing liposomes, Ca²⁺-EDTA-containing liposomes and benzyl alcohol.

15. The method of claim 1, wherein said macrophage inhibiting agent is an endocytosis inhibitor.

16. The method of claim 15, wherein said endocytosis inhibitor is selected from the group consisting of cytochalasin D, colchicine, nocodazole, benzyl alcohol, an anaesthetic, an agent that sequesters and/or complexes with cholesterol and a cationic amphiphile.

17. The method of claim 1, wherein said macrophage inhibiting agent is a lysomotropic agent.

18. The method of claim 17, wherein said lysomotropic agent is selected from the group consisting of monensin, chloroquine and ammonium chloride.

19. The method of claim 1, wherein a sustained release composition comprises said biologically active agent.

20. The method of claim 1, wherein said biologically active agent is selected from the group consisting of a small molecule, a protein, a peptide, a peptidomimetic and a nucleic acid.

21. The method of claim 1, wherein said biologically active agent is a protein or a peptide.

22. The method of claim 21, wherein said protein or a peptide is selected from the group consisting of an immunoglobulin, insulin and growth hormone.

23. The method of claim 1, wherein for a formulation comprising said biologically active agent, said biologically active agent has a rate of transport through the cell linings of a pulmonary system (K(1)) that is greater than the rate of degradation of said biologically active agent in the pulmonary system (K(2)).

24 The method of claim 23, wherein K(1) is at least 5 times greater than K(2).

25. The method of claim 23, wherein said biologically active agent has a molecular weight that is equal to or greater than 5,000 Daltons.

26. The method of claim 1, wherein said biologically active agent has a local biological effect in the pulmonary system.

27. A method of enhancing pulmonary absorption of a biologically active agent comprising administering to a pulmonary system of a subject:
a) a biologically active agent; and
b) a macrophage inhibiting agent suitable for administration to a pulmonary system,
wherein said macrophage inhibiting agent is present in an amount sufficient to enhance bioavailability of said biologically active agent.

28. A method of enhancing bioavailability of a biologically active agent that is administered to the pulmonary system of a subject, comprising:
a) determining for a formulation comprising a biologically active agent:
i) the rate of transport of said biologically active agent through the cell linings of a pulmonary system (K(1)); and
ii) the rate of degradation of said biologically active agent in the pulmonary system (K(2)); and
b) administering a dose of said biologically active agent, wherein K(1) is at least 5 times greater than K(2).

29. The method of claim 28, wherein:
a) transport of said biologically active agent through the cell linings of a pulmonary system is a saturable process, and K(1) is the rate of transport of said biologically active agent through the cell linings of a pulmonary system at saturation; and
b) said method further comprises:
i) determining the rate of release of said biologically active agent from a delivery vehicle (K(0)); and
ii)) administering a dose of said biologically active agent wherein K(0) is less than or equal to K(1).

30. The method of claim 29, wherein K(0) is minimized by administering a sustained release composition comprising said biologically active agent.

31. The method of claim 28, wherein K(2) is reduced by administering a macrophage inhibiting agent suitable for administration to a pulmonary system.

32. The method of claim 31, wherein said macrophage inhibiting agent is a bisphosphonate compound.

33. The method of claim 32, wherein said bisphosphonate compound is dichloromethylene diphosphonate.

34. The method of claim 29, wherein:
a) K(2) is reduced by administering a macrophage inhibiting agent suitable for administration to a pulmonary system;
b) K(0) is minimized by administering a sustained release composition comprising said biologically active agent; and
c) K(1) is increased by:
i) administering one or more phospholipids, surfactants and/or excipients to the pulmonary system of said subject; and/or
ii) administering phosphate buffered saline to the pulmonary system of said subject.

35. A method of determining an optimal dosing regimen for pulmonary delivery of a biologically active agent comprising:
a) determining for a formulation comprising a biologically active agent:
i) the rate of transport of said biologically active agent through the cell linings of a pulmonary system (K(1)); and
ii) the rate of degradation of said biologically active agent in the pulmonary system (K(2)); and
b) selecting a dosing regimen of said biologically active agent, wherein K(1) is at least 5 times greater than K(2).

36. The method of claim 35 further comprising:
a) determining the rate of release of said biologically active agent from a delivery vehicle (K(0)); and
b) administering a dose of said biologically active agent wherein K(0) is less than or equal to K(1).

37. A method of conducting and optimizing dosing for pulmonary delivery of a biologically active agent comprising:
a) determining:
i) the rate of transport through the cell linings of a pulmonary system (K(1)); and
ii) the rate of degradation of said biologically active agent in the pulmonary system (K(2)); and
b) selecting a dosing regimen of said biologically active agent, wherein K(1) is at least 5 times greater than K(2).

38. The method of claim 37 further comprising:
a) determining the rate of release of said biologically active agent from a delivery vehicle (K(0)); and
b) administering a dose of said biologically active agent wherein K(0) is less than or equal to K(1).

39. The method of claim 37 further comprising formulating a pharmaceutical preparation comprising said biologically active agent at a dose comporting with said selected dosing regimen.

40. A method of administering a therapeutically active agent to the pulmonary system of a subject comprising administering:
a) a therapeutically active agent; and
b) a macrophage inhibiting agent suitable for administration to a pulmonary system.

41. The method of claim 40 wherein said macrophage inhibiting agent is present in an amount sufficient to enhance bioavailability of said biologically active agent.
